(19) United States
(12) Patent Application Publication
(10) Pub. No.: US 2004/0002543 A1
(43) Pub. Date: Jan. 1, 2004

(54) Compositions comprising GABA analogs and a decongestant to relieve sinus headache pain

(76) Inventors: Leslie Magnus, Livingston, NJ (US); Catherine Segal, Chester, NJ (US)

Correspondence Address: WARNER-LAMBERT COMPANY, 2800 PLYMOUTH RD, ANN ARBOR, MI, 48105, US

(21) Appl. No.: 10/610,386

(22) Filed: Jun. 30, 2003

Related U.S. Application Data
(63) Continuation of application No. 09/743,433, filed on Feb. 16, 2001, now abandoned.
Application No. 09/743,433 is a 371 of international application No. PCT/US99/13946, filed on Jun. 18, 1999.

(51) Int. Cl. A61K 31/195

(52) U.S. Cl. 514/561

(57) ABSTRACT

This invention is composition and method for treating sinus headache or sinus pains including analogs of glutamic acid and gamma-aminobutyric acid in combination with a decongestant.

### BACKGROUND OF THE INVENTION

1. Field of the Invention

The present invention relates to compositions comprising analogs of glutamic acid and gamma-aminobutyric acid (GABA) in combination with a decongestant for the treatment of sinus headache pain.

2. Description of Related Art

GABA analogs are known agents useful in antiseizure therapy for central nervous system disorders such as epilepsy, Huntington's chorea, cerebral ischemia, Parkinson's disease, tardive dyskinesia, and spasticity. It has also been suggested that the compounds can be used as antidepressants, anxiolytics, and antipsychotics. See WO 92/09560 (U.S. Ser. No. 618,692 filed Nov. 27, 1990) and WP 93/23383 (U.S. Ser. No. 886,080 filed May 20, 1992).

WO 97/33858 teaches that compounds related to gabapentin are useful or treating epilespy, faintness attacks, hypokinesia, cranial disorders, neurodegenerative disorders, depression, anxiety, panic, pain, and neuropathological disorders. WO 97/33858 does not specify what forms of pain are treated.

Additionally, the compounds of the invention are known for treatment of neuropathic pain. For example, see Rosner H; Rubin L; Kestenbaum A., Gabapentin adjunctive therapy in neuropathic pain states. Clin J Pain, 1996 Mar, 12:1, 56-8; Segal A Z; Rordorf G., Gabapentin as a novel treatment for postherpetic neuralgia. Neurology, 1996 Apr, 46:4, 1175-6; Wetzel C H; Connelly J F., Use of gabapentin in pain management. Ann Pharmacother, 1997 Sep, 31:9, 1082-3; Zapp J J., Postpoliomyelitis pain treated with gabapentin [letter]. Am Fam Physician, 1996 Jun, 53:8, 2442, 2445; Cheville A, et al., Neuropathic pain in radiation myelopathy:a case report. Program book, American Pain Society (14th Annual Scientific Meeting). Abstract #95823, p. A-115; Sist T; Filadora V; Miner M; Lema M., Gabapentin for idiopathic trigeminal neuralgia: report of two cases. Neurology, 1997 May, 48:5, 1467; Waldman S D, Tutorial 28: Evaluation and Treatment of Trigeminal Neuralgia. Pain Digest (1997) 7:21-24; Mellick L B; Mellick G A., Successful treatment of reflex sympathetic dystrophy with gabapentin [letter]. Am J Emerg Med, 1995 Jan, 13:1, 96; Mellick G A; Seng M I., The use of gabapentin in the treatment of reflex sympathetic dystrophy and a phobic disorder. Am J Pain Manage 1995; 5:7-9; Mellick G A; Mellicy L B; Mellick L B., Gabapentin in the management of reflex sympathetic dystrophy [letter]. J Pain Symptom Manage, 1995 May, 10:4, 265-6; Mellick G A; Mellick L B., Reflex sympathetic dystrophy treated with gabapentin. Arch Phys Med Rehabil, 1997 Jan, 78:1, 98-105 and Mackin G A., Medical and pharmacologic management of upper extremity neuropathic pain syndromes. J Hand Ther, 1997 April-June, 10:2, 96-109.

Sinus headaches result from the inflammation of the sinus linings resulting in sinus pressure and pain. Sinus headaches are a common problem for adults over the age of 35 year and are often precipitated by the common cold or allergic rhinits. Common treatments include analgesics and decongestants.

Until the present invention, there has not been any report of using GABA analogs, which were originally used for treating the neurological disorder epilepsy, and has been further used to treat neuropathic or neurologically based pain, in combination with decongestants and other sinus medications to relieve sinus headache or sinus pain.

### SUMMARY OF THE INVENTION

This invention provides a method for treating sinus headache comprising administering to a subject suffering from sinus headache an effective amount of a GABA analog in combination with a decongestant. A preferred GABA analog utilizes a cyclic amino acid compound of Formula I

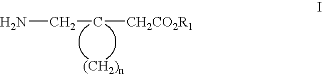

wherein R₁ is hydrogen or lower alkyl and n is an integer of from 4 to 6, and the pharmaceutically acceptable salts thereof. An especially preferred embodiment utilizes a compound of Formula I where R₁ is hydrogen and n is 4, which compound is 1-(aminomethyl)-cyclohexane acetic acid, known generically as gabapentin.

The decongestants useful in the present invention include various sympathomimetic drugs such as pseudoephedrine, ephedrine, phenylephrine and pharmaceutically acceptable salts thereof. An additional decongestant useful in the present invention is the complementary medicine ma huong, which is known for its decongestant properties. These decongestants are known to those skilled in the art as therapeutic agents effective in the relief of nasal congestion.

In another embodiment, the invention includes treating sinus headache or sinus pain with a compound of Formula II in combination with a decongestant. Formula II is

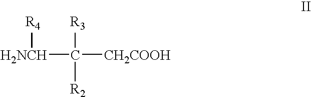

wherein R₂ is a straight or branched alkyl of from 1 to 6 carbon atoms, phenyl, or cycloalkyl having from 3 to 6 carbon atoms; R₃ is hydrogen or methyl; and R₄ is hydrogen, methyl, or carboxyl; or an individual enantiomeric isomer thereof; or a pharmaceutically acceptable salt thereof, in unit dosage form, to a mammal in need of said treatment.

Preferred compounds of the invention are those wherein R₄ and R₃ are hydrogen, and R₂ is —(CH₂)₀₋₂-i C₄H₉ as an (R), (S), or (R,S) isomer.

The more preferred compounds of Formula II invention are (S)-3-(aminomethyl)-5-methylhexanoic acid and 3-aminomethyl-5-methyl-hexanoic acid, now known generically as pregabalin.

### DETAILED DESCRIPTION OF THE PREFERRED EMBODIMENTS

The method of this invention utilizes any GABA analog. A GABA analog is any compound derived from or based upon gamma-aminobutyric acid. The compounds are readily available, either commercially, or by synthetic methodology well-known to those skilled in the art of organic chemistry. The preferred GABA analogs to be utilized in the method of this invention are cyclic amino acids of Formula I. These are described in U.S. Pat. No. 4,024,175, which is incorporated herein by reference. Another preferred method utilizes the GABA analogs of Formula II, and these are described in U.S. Pat. No. 5,563,175 which is incorporated herein by reference.

All that is required to practice the method of this invention is to administer a GABA analog and a decongestant in an amount that is effective to treat the sinus headache or sinus pain. The amount of the GABA analog will generally be from about 1 to about 300 mg per kg of subject body weight. Typical doses will be from about 10 to about 5000 mg per day for an adult subject of normal weight. It is expected that common doses that might be administered could be from 100 mg three times a day up to 600 mg four times a day. Commercially available capsules of 100 mg, 300 mg and 400 mg of gabapentin can be administered. Alternate forms include liquids and film-coated tablets.

If a compound of Formula II, such as pregabalin is used, the dosage level is one sixth that of gabapentin. The dosage range for pregabalin is from about 0.15 mg to about 50 mg per kg per day of subject body weight. Typical dosages for pregabalin will be from about 1.6 mg to about 840 mg per day with individual dosages ranging from abut 0.15 mg to about 65 mg per dose.

A therapeutically effective decongestant amount of a sympathomimetic drug is that amount which produces the desired decongestant therapeutic response upon oral administration and can be readily determined by one skilled in the art by the use of conventional techniques and by observing results obtained under analogous circumstances. In determining the therapeutically effective amount, a number of factors are considered, including but not limited to: the particular compound administered; the bioavailability characteristics of the pharmaceutical composition administered; the dose regimen selected; and other relevant circumstances.

A therapeutically effective decongestant amount of a sympathomimetic drug will vary from about 1 mg to about 200 mg. Preferred amounts will vary from about 5 mg to about 150 mg.

These sympathomimetic drugs are generally effective when administered orally in unit dosage form on a four times a day dosage schedule wherein the unit dosage form provides immediate-release of the active medicament. For example, the recommended dosage for pseudoephedrine hydrochloride in adults is 60 mg every 6 hr (q.i.d.). In addition, unit dosage forms containing sympathomimetic drugs can be formulated to provide prolonged release of the active medicament so as to allow the effective daily dose to be administered on a less frequent dosage schedule. For example, the recommended dosage for pseudoephedrine hydrochloride in a prolonged-release formulation can be 120 mg. b.i.d.

In addition to the GABA analog and decongestant, the compositions according to the present invention can be administered concomitantly with antihistamines for relief of nasal congestion associated with allergic rhinitis. Such antihistamines include, but are not limited to, the following: chlorpheniramine, brompheniramine, dexchlorpheniramine, dexbrompheniramine, tripolidine, diphenhydramine, doxylamine, tripelennamine, cyproheptadine, carbinoxamine, bromodiphenhydramine, phenindamine, pyrilamine, azatadine, terfenadine, astemizole, loratadine, acrivastine, cetirizine, azalastine, evastine, levocabastine, and pharmaceutically acceptable salts thereof.

The compositions of the present invention can also include a second pain reliever. These pain relievers include analgesics such as aspirin, acetaminophen, ibuprofen, flurbiprofen, naproxen, mefenamic acid, ketoprofen, indomethacin, indoprofen, azapropazone, dicclofenac, difluisal, fenbufen, fenoprofen, piroxicam, sulindac, suprofen, tiaprofenic acid, tolmetin, droxicam, meloxicam, tenoxicam, etodolac, oxindanac or mixtures thereof.

The compositions of the present invention can also include an antitussive such as, but not limited to, the following: dextromethorphan, codeine, terpin hydrate and pharmaceutically acceptable salts thereof.

The compositions of the present invention can also include an expectorant. The expectorants useful in the present invention include, but are not limited to, the following: guaifenesin, potassium guaicolsulfonate, potassium iodide, potassium citrate, iodinated glycerol, acetylcysteine, carboxymethylcysteine, ambroxol, sobrerol, and pharmaceutically acceptable salts thereof.

The GABA compounds of the present invention may form pharmaceutically acceptable salts with both organic and inorganic acids or bases. For example, the acid addition salts of the basic compounds are prepared either by dissolving the free base in aqueous or aqueous alcohol solution or other suitable solvents containing the appropriate acid and isolating the salt by evaporating the solution. Examples of pharmaceutically acceptable salts are hydrochlorides, hydrobromides, hydrosulfates, etc. as well as sodium, potassium, and magnesium, etc. salts.

The compounds of Formula II can contain one or several asymmetric carbon atoms. The invention includes the individual diastereomers or enantiomers, and the mixtures thereof. The individual diastereomers or enantiomers may be prepared or isolated by methods already well-known in the art.

Pharmaceutical compositions of the compound of the present invention or its salts are produced by formulating the active compound in dosage unit form with a pharmaceutical carrier. Some examples of dosage unit forms are tablets, capsules, pills, powders, aqueous and nonaqueous oral solutions and suspensions, and parenteral solutions packaged in containers containing either one or some larger number of dosage units and capable of being subdivided into individual doses. Some examples of suitable pharmaceutical carriers, including pharmaceutical diluents, are gelatin capsules; sugars such as lactose and sucrose; starches such as corn starch and potato starch, cellulose derivatives such as sodium carboxymethyl cellulose, ethyl cellulose, methyl cellulose, and cellulose acetate phthalate; gelatin; talc; stearic acid; magnesium stearate; vegetable oils such as peanut oil, cottonseed oil, sesame oil, olive oil, corn oil, and oil of theobroma; propylene glycol, glycerin; sorbitol; polyethylene glycol; water; agar; alginic acid; isotonic saline, and phosphate buffer solutions; as well as other compatible substances normally used in pharmaceutical formulations. The compositions of the invention can also contain other components such as coloring agents, flavoring agents, and/or preservatives. These materials, if present, are usually used in relatively small amounts. The compositions can, if desired, also contain other therapeutic agents.

The percentage of the active ingredients in the foregoing compositions can be varied within wide limits, but for practical purposes it is preferably present in a concentration of at least 10% in a solid composition and at least 2% in a primary liquid composition. The most satisfactory compositions are those in which a much higher proportion of the active ingredient is present.

Routes of administration of the GABA analogs or their salts are oral or parenteral. For example, a useful intravenous dose is between 5 and 50 mg of GABA analog and a useful oral dosage of GABA analog is between 20 and 800 mg. The dosage is within the dosing range used in treatment of pain or as would be with the needs of the patient as described by the physician.

The advantages of using the compounds of Formula I and II, especially gabapentin and pregabalin, in the instant invention include the relatively nontoxic nature of the compounds, the ease of preparation, the fact that the compounds are well-tolerated, and the ease of IV administration of the drugs. Gabapentin has few interactions with major classes of drugs since it is not metabolized in the liver, but rather excreted unchanged from the body. Further, the drugs are not metabolized in the body. The subjects treated with the method of the present invention are mammals, including humans.

We claim:

1. A method for treating sinus headache or sinus pain, comprising administering a pharmaceutical composition comprising:
(a) an analgesically effective amount of a GABA analog; and
(b) an effective amount of a decongestant.

2. The method according to claim 1, wherein the GABA analog is the compound according to Formula I: 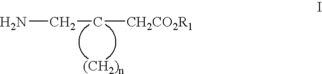
wherein R₁ is hydrogen or lower alkyl and n is an integer of from 4 to 6, and the pharmaceutically acceptable salts thereof.

3. The method according to claim 2, wherein Formula I comprises gabapentin.

4. The method according to claim 1, wherein the decongestant is selected from the group consisting of pseudoephedrine, ephedrine, phenylephrine, pharmaceutically acceptable salts thereof and ma huong.

5. The method according to claim 2, comprising from about 10 mg to about 400 mg of Formula I.

6. The method according to claim 3, comprising from about 10 mg to about 400 mg of gabapentin.

7. The method according to claim 3, comprising from about 10 mg to about 400 mg of gabapentin and from about 60 mg to about 200 mg of decongestant.

8. The method according to claim 1, wherein the GABA analog is a compound according to Formula II: 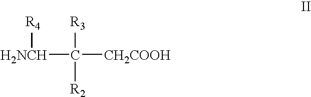
wherein R₂ is a straight or branched alkyl of from 1 to 6 carbon atoms, phenyl, or cycloalkyl having from 3 to 6 carbon atoms; R₃ is hydrogen or methyl; and R₄ is hydrogen, methyl, or carboxyl.

9. The method according to claim 8, wherein Formula II comprises pregabalin.

10. The method according to claim 8, comprising from about 0.15 mg to about 65 mg of Formula II.

11. The method according to claim 9, comprising from about 0.15 mg to about 65 mg of pregabalin.

12 A composition for eliciting an enhanced analgesic response in a mammal comprising:
(a) an analgesically effective amount of a GABA analog; and
(b) an effective amount of a decongestant.

13. The composition according to claim 12, wherein the GABA analog the compound according to Formula I: 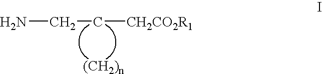
wherein R₁ is hydrogen or lower alkyl and n is an integer of from 4 to 6, and the pharmaceutically acceptable salts thereof.

14. The composition method according to claim 13, wherein Formula I comprises gabapentin.

15. The composition according to claim 13, comprising from about 10 mg to about 400 mg of Formula I.

16. The composition according to claim 14, comprising from about 10 mg to about 400 mg of gabapentin.

17. The composition according to claim 12, wherein the GABA analog is a compound according to Formula II: 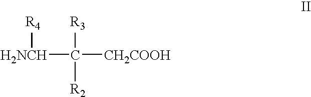
wherein R₂ is a straight or branched alkyl of from 1 to 6 carbon atoms, phenyl, or cycloalkyl having from 3 to 6 carbon atoms; R₃ is hydrogen or methyl; and R₄ is hydrogen, methyl, or carboxyl.

18. The composition according to claim 17, wherein Formula II comprises pregabalin.

19. The composition according to claim 17, comprising from about 0.15 mg to about 65 mg of Formula II.

20. The composition according to claim 19, comprising from about 0.15 mg to about 65 mg of pregabalin.
